(19) United States
(12) Patent Application Publication
(10) Pub. No.: US 2005/0267319 A1
(43) Pub. Date: Dec. 1, 2005

(54) Brachytherapy seed loader and containers

(76) Inventors: Jack C. White, Alpharetta, GA (US); Stephen N. Carr, Flowery Branch, GA (US)

Correspondence Address: KEVIN J. DUNLEAVY;KNOBLE YOSHIDA & DUNLEAVY, LLC, EIGHT PENN CENTER, SUITE 1350, 1628 JOHN F. KENNEDY BOULEVARD, PHILADELPHIA, PA, 19103, US

(21) Appl. No.: 11/126,581

(22) Filed: May 11, 2005

Related U.S. Application Data
(60) Provisional application No. 60/570,209, filed on May 12, 2004.

(51) Int. Cl. A61N 5/00; A61M 36/00

(52) U.S. Cl. 600/7

(57) ABSTRACT

A seed and/or spacer loader and containers for storage and dispensing of brachytherapy seeds and spacers are disclosed. The seed and/or spacer loader has a holding area to hold seeds and/or spacers prior to loading. The seed and/or spacer loader has a loading mechanism that loads seeds and/or spacers into a receiving device, such as a needle or strand. The seed and/or spacer loader further includes a selection mechanism for selectively dispensing seeds and/or spacers from different containers. The containers have an advancing strip mechanism or a belt which can be employed to position seeds and/or spacers for dispensing from the container and to index the next seed or spacer to a dispensing position.

### CROSS-REFERENCE TO RELATED APPLICATIONS

This application claims the benefit of U.S. provisional patent application No. 60/570,209, filed on May 12, 2004, pursuant to 35 U.S.C. §119(e).

### BACKGROUND OF THE INVENTION

1. Field of the Invention

The present invention relates to a devices for storage and shipping of brachytherapy seeds and/or spacers, devices for loading seeds and spacers into brachytherapy devices such as implantation needles and seed strands, and to methods of using such devices either alone or in combination.

2. Description of the Related Technology

Brachytherapy is the treatment of the body with radioactive materials for the purpose of therapy. Seeds containing radioactive material are used in brachytherapy to treat a variety of conditions, such as prostate cancer, for example. One method for placement of radioactive seeds in the body for brachytherapy is implantation of the seeds and, optionally, spacers using one or more implantation needles. In order to prepare such implantation needles for use, the needle must first be loaded with seeds and, optionally, spacers. Often, loading of needles is performed at the site of the brachytherapy treatment, in which case, one or more containers containing seeds or spacers are shipped to the treatment site where the seeds and, optionally, spacers, are then loaded into the implantation needle. This may be accomplished using a seed loader, or can be done manually. When using a seed loader the seeds are transferred from the shipping container into one or more implantation needles by the seed loader. A variety of seed loaders exist for this purpose.

U.S. Patent Application Publication No. 2002/0058854 to Reed discloses a seed loader. In one embodiment of the seed loader, two magazines are employed, one for spacers and one for seeds. Two different plungers are employed to select the dispensing of seeds or spacers. A transparent portion of the seed loader can be used to visually verify the arrangement of seeds and spacers before loading them into the implantation needle.

U.S. Pat. No. 6,537,192 to Elliot et al. discloses an automated radioisotope seed loader for loading radioactive seeds into implantation needles. This device employs a rotatable drum with holes or chambers located around the periphery for holding radioactive seeds prior to loading into implantation needles. Elliot et al. contemplates shipping the seeds to the treatment site in the rotatable drum, or, alternatively, the rotatable drum can be loaded at the treatment site using a separate loading clip. When using the loading clip, loose seeds are loaded into the loading clip and the loose seeds can be placed in the loading clip in an arrangement dictated for a particular treatment, prior to shipping the seeds to the treatment site.

The device of Elliot et al. employs a push rod to transfer one seed at a time from the loading clip into the chambers of the rotatable drum. The loading clip is first positioned in the device of Elliot et al. and then the user, via a handle, actuates the push rod. The drum loading process can alternatively be automated. It is also mentioned that two loading clips can be used in loading the cartridge, one filled with spacers, the other with seeds. Additionally, each loading clip can be provided with indication means for indicating the contents of the loading clip. In one embodiment, the loading clip is provided with a machine-readable storage medium that stores information about the contents of the loading clip.

U.S. Pat. No. 6,629,960 to Fontayne discloses a needle hub for loading implantation needles from a seed cartridge. The actuation of a trigger on the needle hub activates a pusher or stylet to push a single seed out of the seed cartridge directly into an implantation needle. A seed counter counts the seeds that have been loaded. The seed counter is viewable through a window and includes a lens that helps to provide a clear view of the counter. Since the seeds are transferred directly from the seed cartridge to the implantation needle, this device does not permit the user to view the seed and spacer arrangement prior to loading the needle, which increases the risk of error in loading the implantation needles.

Med-Tec offers a 120-seed drum cartridge, which cartridge is described on Med-Tec's web site at, http://www.additec.de/medtec/products/brachy/cart.htm. The drum cartridge is a shielded cartridge that holds up to 120 seeds, with an indicator that displays the total number of seeds and is designed for storage and shipping of seeds to the treatment site for loading into a plurality of implantation needles.

Mentor Corporation offers a SeedVue™ device which is described on the Mentor web site at http://www.mentorcorp.com/cancer/prostatecancer/physicianlPC_seedvue.htm. The SeedVue™ device is a handheld brachytherapy magazine. The seeds contained in the SeedVue™ device can be loaded into implantation needles using a loading device, such as the Isoloaderm from Mentor Corporation. The SeedVue™ device has a transparent body that enables visualization of the seeds from any angle. The device includes a spring mechanism for advancing the seeds prior to loading them into the needle. The SeedVue™ device is also provided with a drop-down shield to provide shielding of the radioactive material prior to and during the loading process and includes an indicator to indicate the number of seeds remaining in the device at any given time.

The devices discussed above provide a variety of means for transferring brachytherapy seeds and/or spacers into implantation needles. However, certain of these devices have a risk of seeds being loaded incorrectly. In addition, several of these devices are bulky, unnecessarily complex and may be difficult to properly sterilize for use. Finally, some of these devices load loose seeds in a manner which risks jamming of the seeds in the loading device. Therefore there remains a need for improved apparatus for shipping brachytherapy seeds to a treatment site and for improved devices for loading seeds and spacers into an implantation needle.

Another method for delivery of brachytherapy seeds to the body involves first positioning a plurality of seeds and, optionally, spacers into a brachytherapy seed strand and then implanting the seed strand in the body at the desired location. Various types of seed strands are known in the art from, for example, Scott, W. P., “Surgical Radiation Therapy With Vicryl™ ¹²⁵I Absorbable Sutures,” Surg. Gynecol. Obstet., 1976; 142:667-670.

Typically, such seed strands are loaded with seeds and, optionally, spacers, prior to shipping to the treatment site. However, in order to provide maximum flexibility for a particular treatment, it is desirable to provide a device, which permits seed strands to be loaded in a reliable manner at the treatment site.

Accordingly, the present invention provides various devices for storage and shipment of brachytherapy seeds and/or spacers. In other embodiments, the present invention provides various devices for loading brachytherapy seeds into implantation needles, seed strands, or the like, at the treatment site.

### SUMMARY OF THE INVENTION

In one embodiment, the present invention relates to devices for storage and shipment of brachytherapy seeds, and, optionally, spacers to a treatment site. The devices of the present invention may be employed securely position the seeds and spacers for shipment to the treatment site in a manner whereby the exact location of a particular seed or spacer is known, thereby facilitating customization of a particular brachytherapy treatment. In order to accomplish this, the storage and shipment devices of the present invention are provided with various apparatus for securely positioning and holding the seeds, and, optionally, spacers during shipment, and to reliably advance the seeds or spacers into position for loading into an implantation needle, seed strand or other brachytherapy seed implantation devices.

In another embodiment, the present invention provides a hand-held seed and spacer-loading device for loading seeds and/or spacers into implantation needles, seed strands or other brachytherapy seed implantation devices. The loading device is adapted for loading from brachytherapy seed or spacer shipping containers. The loading device includes a mechanism for transferring individual seeds and/or spacers from such shipping containers to a holding area to hold an arrangement of seeds and/or spacers prior to loading the seeds and/or spacers into an implantation device. The loading device also includes apparatus for transferring the desired arrangement of seeds and/or spacers from the holding area into an implantation device.

The present invention also relates to methods of transferring brachytherapy seeds and, optionally, spacers, from a shipping container to a loading device and to methods of loading various implantation devices with brachytherapy seeds and/or spacers.

For a better understanding of the invention, its advantages, and the objects obtained by its use, reference should be made to the drawings which form a further part hereof, and to the accompanying descriptive matter, in which there is illustrated and described various specific embodiments of the present invention.

### BRIEF DESCRIPTION OF THE DRAWINGS

FIG. 1A is a perspective view of a seed and/or spacer shipping container in accordance with one embodiment of the present invention.

FIG. 1B is a perspective view of the shipping container of FIG. 1A viewed from the opposite side of that shown in FIG. 1A.

FIG. 1C is a cross-sectional view of the shipping container of FIGS. 1A-1B.

FIG. 2 is a cross-sectional view of an alternative embodiment of a spring-loaded seed and/or spacer shipping container in accordance with the present invention.

FIG. 3A is a perspective view of an alternative container for storage and shipping of seeds and/or spacers in accordance with the present invention shown in a shipping configuration.

FIG. 3B is a perspective view of a portion of the container of FIG. 3A in an unwound position.

FIG. 3C is a partial cross-sectional view showing one seed or spacer holder of the container of FIGS. 3A-3B.

FIG. 4 is a cartridge adapted for use with the container of FIGS. 3A-3C.

FIG. 5A is view of a hand-held brachytherapy seed and/or spacer-loading device in accordance with one embodiment of the present invention.

FIG. 5B is a view of the device of FIG. 4A with an implantation needle attached to the device and an arrangement of seeds and spacers ready for loading.

FIG. 5C is a view of the device of FIG. 4B actuating for transferring the arrangement of seeds and spacers from the loader to the implantation needle.

FIG. 5D is a cross-sectional view of one embodiment of a loading device in accordance with FIGS. 5A-5C adapted for use with the container of FIGS. 1A-1C.

### DETAILED DESCRIPTION OF EMBODIMENTS OF THE INVENTION

The devices of certain embodiments the present invention are designed for the storage and shipment of brachytherapy seeds and/or spacers to a treatment site in a manner convenient for reliable, verifiable loading into various types of brachytherapy seed implantation devices.

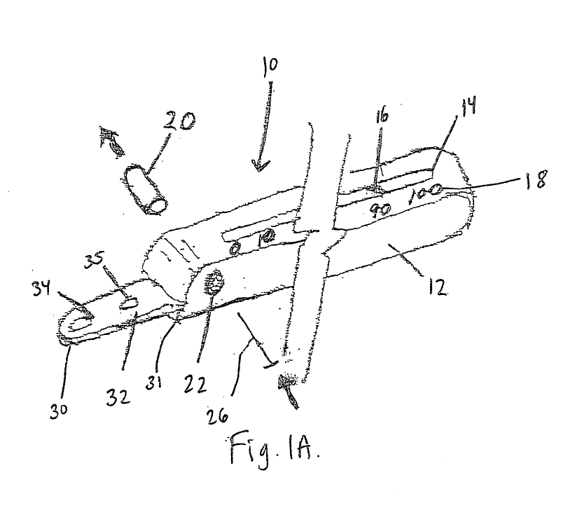

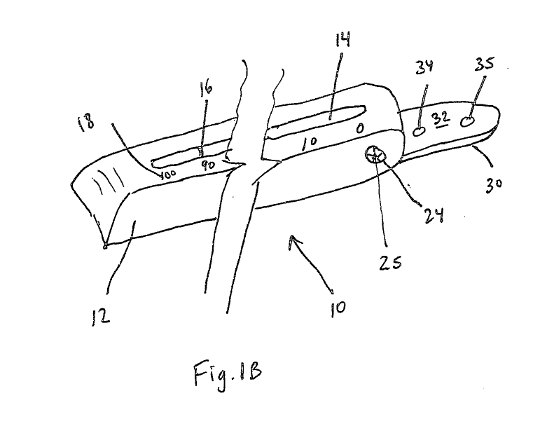

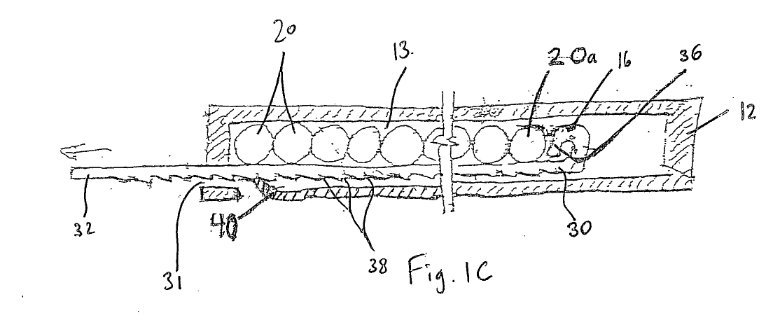

Referring now to the drawings, wherein like reference numerals designate corresponding structure throughout the several views, FIG. 1A shows one embodiment of storage and shipping container 10 for brachytherapy seeds and/or spacers. Container 10 includes a housing 12 which may be fabricated from any suitable material. In one embodiment, housing 12 may be fabricated from a sterilizeable material. Housing 12 may include radiation-shielding material for the purpose of shielding at least some of the radiation emitted by radioactive seeds 20 contained within housing 12. Sufficient radiation shielding material may be used to shield all or a substantial portion of the radiation emitted by seeds 20 in order to provide some degree of protection to persons handling container 10. Housing 12 defines a holding area 13 within which brachytherapy seeds and spacers may be stored. Housing 12 may include a contents-indicator such as a window 14. Window 14 is sufficiently transparent that the user can see the location of either the last seed or spacer 20a located in housing 12 or the location of an indicator 16. Optionally, window 14 contains at least some radiation-shielding material and the material of window 14 may be selected to be sterilizeable.

Housing 12 may be provided with a scale 18 associated with window 14 which forms part of the contents-indicator. Scale 18 may include numeric indicia, as shown, to provide an indication of the number of seeds and/or spacers remaining in container 10, based on, for example, the location of either the last seed or spacer 20a in container 10, or on the location of indicator 16 in container 10. Alternatively, container 10 may employ an electronic indicator instead of a mechanical indicator 16, as shown. Such an electronic indicator could include, for example, a counter for counting the number of seeds and/or spacers which have been dispensed, and a digital display indicating the number of seeds and/or spacers remaining in container 10.

Housing 12 may also include a stylet opening 22 which is aligned with a seed dispensing opening 24 located on the opposite side of housing 12, as shown in FIG. 1B. Stylet opening 22 is adapted to receive a stylet 26 for the purpose of dispensing a seed 20 from container 10 via seed dispensing opening 24. In operation, stylet 26 is inserted into stylet opening 22, engages a seed 20 aligned with seed dispensing opening 24 and pushes the seed 20 out of container 10 through seed dispensing opening 24, as shown in FIG. 1A. Once seed 20 is dispensed via seed dispensing opening 24, stylet 26 is withdrawn via stylet opening 22 and the next seed 20 is positioned in alignment with both stylet opening 22 and seed dispensing opening 24.

Seed dispensing opening 24 may optionally be provided with a retaining structure 25, such as a flexible material with a plurality of slits and including a central opening, as shown. Retaining structure 25 is provided with sufficient rigidity that under the weight of a single seed and/or spacer, and the influence of gravity and/or other external forces generated by handling container 10, a seed and/or spacer is retained in container 10. Retaining structure 25 should, however, be sufficiently flexible to permit dispensing of a seed and/or spacer through retaining structure 25 when force is exerted on the seed or spacer by stylet 26 via stylet opening 22 to dispense the seed or spacer. Other suitable retaining structures 25 may be employed, including, for example, a spring-loaded door or other comparable structure that retains seeds and/or spacers in container 10 until it is desired to dispense them.

Seeds 20 may be positioned for dispensing using advancing strip 30. Advancing strip 30 may be provided with a tab 32 at the leading end of advancing strip 30. Housing 12 is provided with an opening 31 through which advancing strip 30 may be advanced. Tab 32 may be designed for engagement of advancing strip 30 with a pulling mechanism, for the purpose of exerting a pulling force on advancing strip 30. In the embodiment shown in FIG. 1A, tab 32 of advancing strip 30 includes a pair of openings 34, 35 provided for the purpose of engaging with a pulling mechanism. Advancing strip 30 also includes a seed-engaging device 36 located at the trailing end of advancing strip 30. Seed-engaging device 36 may be any suitable mechanical device for transferring a pulling force exerted on tab 32 of advancing strip 30 to the trailing seed 20a in container 20. Exemplary seed-engaging devices 36 include an enlarged trailing end of advancing strip 30, a curled trailing end of advancing strip 30 as shown in FIG. 1C, and an object of suitable size, such as a block or ball, attached to the trailing end of advancing strip 30. In one embodiment, indicator 16 is located on the top of seed-engaging device 36.

Advancing strip 30 may include a series of teeth 38 on the bottom surface thereof, as shown in FIG. 1C. Teeth 38 are adapted to engage with a ratchet mechanism 40 which may be formed, for example, by providing a protruding member on the inside of housing 12, or by cutting a tab into housing 12 and bending the tab upwardly as shown in FIG. 1C. Teeth 38 may be located very close together or may be spaced at a distance approximately equal to one diameter of a seed 20. In either case, the engagement of teeth 38 with ratchet mechanism 40 ensures that advancing strip 30 can only move in the direction it is pulled and not the other direction. In this manner, advancing strip 30 maintains seed 20 in position for dispensing even when no pulling force is being exerted on tab 32 of advancing strip 30.

In operation, a pulling mechanism is attached to tab 32 of advancing strip 30 via openings 34 and/or 35 and a pulling force is exerted on tab 32 of advancing strip 30 to pull seeds 20 in an indexing direction to a point where the first seed 20 is positioned in alignment with stylet opening 22 and seed dispensing opening 24. At this point, the pulling force need not be maintained since the engagement of one surface of one of teeth 38 with a corresponding surface of ratchet mechanism 40 maintains the first seed 20 in position for dispensing by inhibiting movement of advancing strip 30 in a direction opposite the indexing direction. Stylet 26 is inserted into housing 12 via stylet opening 22 and engages one end of seed 20 to thereby push seed 20 out of seed-dispensing opening 24 to dispense a seed. Stylet 26 is then retracted and a advancing strip 30 is pulled until the next seed 20 is positioned for dispensing. As advancing strip 30 is pulled, seed-engaging device 36 pushes the seeds 20 so that the next seed is positioned for dispensing and indicator 16, located on seed-engaging device 36, moves relative to scale 18 to thereby provide an indication of the number of seeds remaining in container 10.

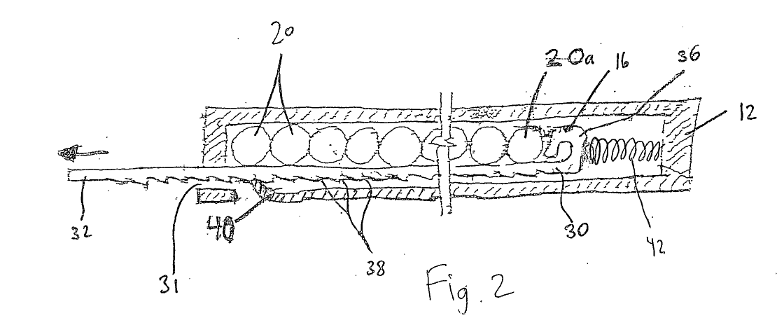

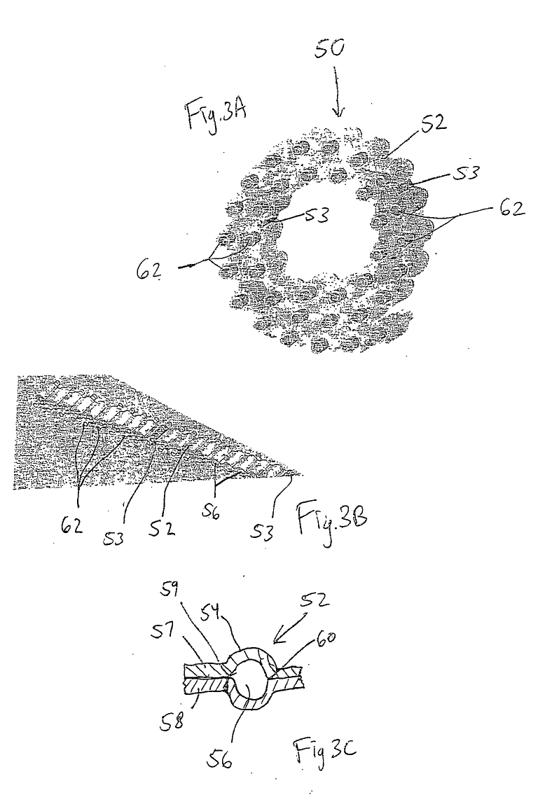

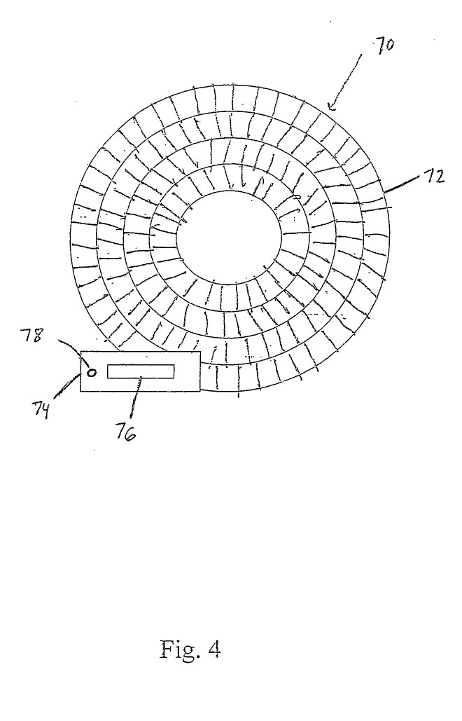

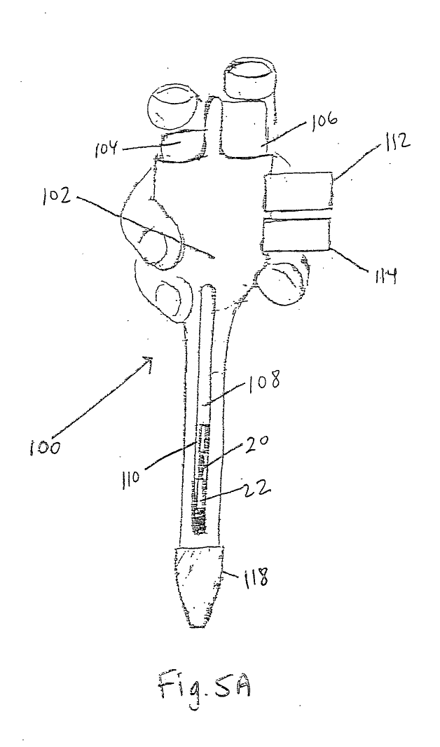

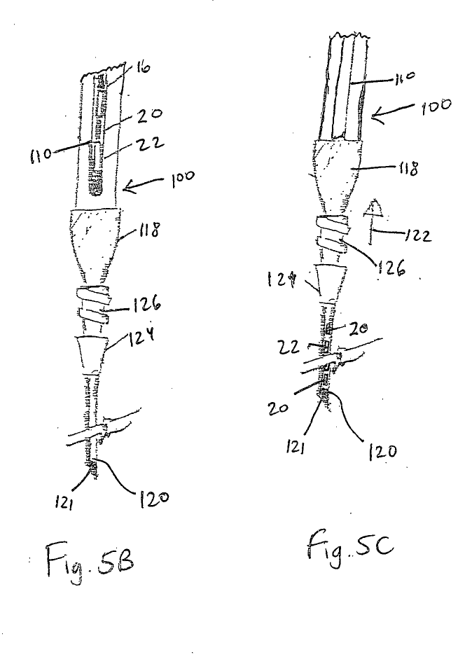

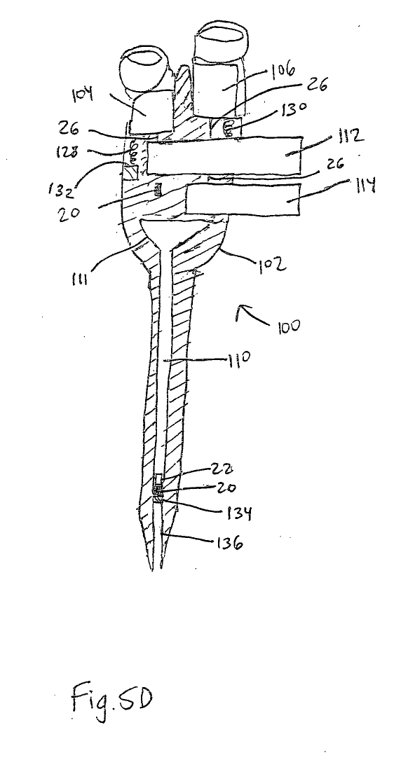

Referring to FIG. 2, there is depicted an alternative embodiment of a container 10 in accordance with the present invention which further includes a spring 42 located in housing 12. One end of spring 42 abuts with housing 12 and the other end of spring 42 abuts with seed-engaging device 36 of advancing strip 30 such that spring 42 exerts a constant force against seed-engaging device 36 of advancing strip 30. In this embodiment, it is not necessary to pull advancing strip 30 via tab 32 since advancement of advancing strip 30 can be accomplished by the force exerted on advancing strip 30 by spring 42. Specifically, when a seed 20 is dispensed out of container 10, the force of spring 42 on seed-engaging device 36 of advancing strip 30 will push seeds 20 to the left to thereby position a new seed 20 in alignment with stylet opening 22 and seed dispensing opening 24. Spring 42 may be attached to one or both of housing 12 and advancing strip 30 by any suitable attachment mechanism. Spring 42 can also be used in combination with a pulling mechanism for exerting a pulling force on tab 32 of advancing strip 30, if desired.

Referring to FIGS. 3A-3B, there is shown an alternative embodiment of a container 50 for storage and shipping of seeds and/or spacers 62. In this embodiment, container 50 is in the form of a bandoleer. Container 50 includes a belt 52 which forms a plurality of seed and/or spacer holders 54 along the length of the belt 52 as shown.

In one embodiment, shown in FIG. 3C, seed and/or spacer holders 54 are formed by two layers 57, 58 of belt 52 which are separated over a short distance to form a space 56 between the belt layers 57, 58. At the beginning 59 and end 60 of each space 56, the two layers 57, 58 of belt 52 are fastened together by any suitable means such as adhesives, stitching, etc. or are formed integrally with one another. In this manner, a space 56 of predetermined size is defined between the two layers 57, 58 of belt 52. Preferably, both ends of each space 56 are open to permit a stylet to be inserted through one end of each space 56 and to permit a seed or spacer 62 to be dispensed through the opposite end of each space 56. In one embodiment, belt 52 is narrower than the axial length of the seeds and/or spacers 62 to permit a portion of the seeds and/or spacers 62 to protrude from one or both ends of each space 56, as shown in FIGS. 3A-3B. In another embodiment, seeds and/or spacers 62 can be completely encapsulated in belt 52, except for one or both ends of the seed and/or spacer 62, in order to provide a more secure hold while at the same time facilitating dispensing.

Belt 52 is preferably fabricated from a flexible material which permits belt 56 to be rolled up as shown in FIG. 3A for storage, transport and use. In the form shown in FIG. 3A, belt 56 takes up a relatively small amount of space, can be contained in a container for use with a seed loading device, and the container 50 can easily be indexed for dispensing the next seed by pulling or pushing the belt 52. Belt 52 may also include indicia 53 thereon for indicating locations on belt 52 or for indicating a specific number of spaces 56 for seeds and/or spacers 62. As shown in FIG. 3B, belt 52 has indicia 53 which indicate every tenth space 56 for a seed and/or spacer 62.

In use, container 50 can be pre-loaded with a pre-determined arrangement of seeds and/or spacers 62 for a particular treatment plan and then the seeds and/or spacers 62 can be dispensed, in the pre-loaded order, into, for example, a seed strand or an implantation needle. Alternatively, container 50 can contain only seeds or only spacers and special loading mechanisms can be employed to transfer the seeds or spacers from container 50 to a seed strand, implantation needle or the like. For example, two containers 50, one containing seeds and another containing spacers, could be used together with the loading device described below to selectively dispense seeds and spacers into a seed strand, implantation needle, or the like.

The bandoleer-shaped container 50 offers the advantage that it can be positioned in various shapes for storage, shipping and use, due to the flexibility of the container. Another advantage of the bandoleer-shaped container 50 is that the seeds and/or spacers are held individually, in a secure manner in a fixed orientation and at a predetermined distance from one another. This can simplify the loading process since it makes it easier to precisely position the seeds and spacers for dispensing and it prevents jamming of the loading device since each seed and/or spacer is held separately from others thereby preventing seeds and/or spacers from becoming misaligned or from being dispensed inadvertently. Also, the bandoleer-shaped container 50 is amenable to high-speed dispensing since the belt 52 can be used for grasping and indexing the container to position seeds and/or spacers in the dispensing position at relatively high throughput rates. Additionally, belt 60 can be sterilized. In addition, product identification information can be printed on belt 60.

Referring to FIG. 4, there is shown a cartridge 70 suitable for use with the bandoleer-shaped container 50. Cartridge 70 may include a housing 72, preferably shaped in a circular or oval manner, as shown, to thereby minimize the longest dimension of housing 72 while still permitting storage of a large number of seeds and/or spacers 62 therein. Bandoleer-shaped container 50 is designed to fit into cartridge 70 in the rolled up position shown in FIG. 3A. Cartridge 70 includes an interface 74 for attaching cartridge 70 to a seed-loading device. Any suitable interface 74 can be used. For example, screw threads, a snap-fit or friction fit mechanism, or other suitable releasable attachment means can be employed to attached cartridge 70 to a seed-loading device. Cartridge 70 may be provided with an indicator such as a window 76 to indicate which seed or spacer is currently in the dispensing position. Window 76 in combination with indicia on belt 52 of bandoleer-shaped container 50 could be employed for this purpose. Cartridge 70 may also include a stylus opening 78 and a corresponding seed dispensing opening, not shown, aligned with the stylus opening 78 for dispensing seeds in the manner described above with respect to the embodiment of FIGS. 1A-1C.

FIG. 5A shows a view of one embodiment of a hand-held seed and/or spacer loader 100 in accordance with the present invention. Seed and/or spacer loader 100 is designed to be held in one hand by a person while loading seeds and/or spacers into a device for delivering brachytherapy seeds and/or spacers to the body, such as an implantation needle, seed strand or the like. Loader 100 includes a housing 102 that is designed to fit in a hand of a person, as shown, for example, in FIG. 5A. Optionally, housing 102 may be provided with one or more contours or indentations, not shown, particularly in the area where housing 102 is grasped, to facilitate grasping and holding loader 100 during use. The grasping area of housing 102 is sized to allow the depression of actuators 104, 106, preferably by the index and middle fingers, as shown in FIG. 5A. Housing 102 may incorporate radiation-shielding material to protect the handler of the device from exposure to radiation emitted from seeds contained in the loader 100. Preferably, housing 102 incorporates sufficient shielding material to shield substantially all radiation emitted from seeds contained in the loader. However, in alternative embodiments, shielding material may be omitted, provided only in certain portions of the housing or the housing may shield only a portion of the emitted radiation. Any conventional radiation shielding material, such as lead or stainless steel, may be employed.

Housing 102 may include a viewing area 108, such as a window made from a sufficiently transparent material to permit viewing of the arrangement of seeds and/or spacers in a holding area 110 defined by housing 102 of loader 100. In one embodiment, the material forming the window may include radiation-shielding material. Viewing area 108 may be employed to visualize the arrangement of seeds 20 and/or spacers 22 in holding area 110 of loader 100 prior to loading seeds 20 and/or spacers 22 into another device. This permits verification of the correct arrangement of seeds 20 and/or spacers 22 in holding area 10 prior to loading into another device. Alternatively, any suitable display device, such as a digital or analog display, may be employed to display information indicative of the layout of seeds 20 and spacers 22 within holding area 110. Holding area 110 is designed to maintain seeds and spacers in a linear, single-file arrangement in the order that seeds and spacers are selectively dispensed from containers into holding area 110. The reason for this is that brachytherapy treatments generally require specific positioning of brachytherapy seeds in the body to provide the desired radiation for treatment. Thus, loader 100 is designed to permit custom loading of seeds and spacers according to a pre-defined treatment plan to help ensure precise positioning of brachytherapy seeds in the body.

In another embodiment, loader 100 may contain a visual or auditory alarm that sounds when the arrangement of seeds and/or spacers in the holding area 110 does not match a predetermined arrangement thereby indicating that the loading area 100 has been loaded incorrectly. In this embodiment, for example, a bar code reader may be connected to processor associated with loader 100 and used to scan a seed and/or spacer arrangement to be loaded. After loading the seeds and/or spacers into holding area 110 of loader 100, the processor then verifies the arrangement of seeds 20 and spacers 22 in holding area 28 and correlates it to the arrangement indicated by the scanned bar code. If the arrangement of seeds and/or spacers in the holding area 110 is incorrect, an alarm is triggered.

Loader 100 is also provided with a tip 118 for connecting loader 100 to a receiving device, such as an implantation needle 120. Tip 118 may include an opening 119 in the distal end of tip 118 for insertion of a portion of the receiving device, preferably the proximal end of an implantation needle 120. Opening 119 preferably provides a friction fit or other suitable, releasable engagement between loader 100 and a portion of a receiving device, again preferably the proximal end of an implantation needle 120. Tip 118 may be formed as part of housing 102, or may be a separate element which fits on the outside of housing 102, by, for example, a friction fit, as shown. In either event, it is desirable that tip 118 be movable relative to housing 102 to permit transfer of seeds and/or spacers from holding area 110 into the receiving device, as shown, for example, in FIG. 5C. From FIGS. 5B-5C, it can be seen that tip 118 moves upwardly relative to housing 102 to permit a proximal end of the receiving device to move into position for receiving seeds and/or spacers from holding area 110 of loader 100.

Still referring to FIG. 5A, containers 112, 114 may be connected to loader 100, as shown. Preferably, one of containers 112, 114 contains radioactive seeds and the other of containers 112, 114 contains spacers since this permits custom loading using loader 100 by selectively loading seeds and/or spacers into holding area 110 using a selection mechanism which functions to allow selective dispensing of seeds and/or spacers from containers 112, 114 into holding area 110. In this embodiment, the selection mechanism includes actuators 104, 106 positioned on housing 102 for single finger actuation when loader 100 is held in one hand, as shown. The selection mechanism also includes apparatus for selectively dispensing seeds and/or spacers from containers 112, 114 responsive to actuation of actuators 104, 106. When one of actuators 104, 106 is depressed, a seed or spacer is dispensed into holding area 110. The selection mechanism permits a handler to effectively select the desired arrangement of seeds 20 and spacers 22 dispensed into holding area 110 for eventual loading into another device. Actuators 104, 106 could also be in the form of triggers, electronic buttons, a touch pad, or other conventional actuating devices that can be operated by the pressure of a finger.

FIG. 5B shows seed and/or spacer loader 100 engaged with a receiving device, in this case an implantation needle 120. Alternatively, the receiving device could be stranding material or any other device into which it is desirable to load seeds and/or spacers. Tip 118 of the seed and/or spacer loader 100 is placed inside a distal end of needle 120 and secured. A compression mechanism operates to release seeds 20 and spacers 22 into needle 30 when tip 118 is compressed against the distal end of needle 120. After tip 118 is secured to the distal end of needle 120, seed and/or spacer loader 100 is pressed in the direction of needle 120. The downward force of the pressing activates the compression mechanism to expand tip 118 so that seeds 20 and spacers 22 fall from the holding area 28 into needle 120. The downward pressure on tip 118 is removed and tip 118 is released from the distal end of needle 120. Needle 120 may then be used for implantation.

Referring to FIGS. 5B and 5C, the movement of loader 100 from a first position wherein seeds and/or spacers are contained in holding area 110 (FIG. 5B) to a second position wherein seeds and/or spacers are dispensed into, for example, an implantation needle 120 (FIG. 5C) is shown. As can be seen in FIG. 5C, tip 118 moves upwardly in the direction of arrow 122, relative to loader 100, from a holding position, shown in FIG. 5B, to a dispensing position shown in FIG. 5C to permit dispensing of seeds and/or spacers from holding area 110 into a receiving device such as an implantation needle 120. The distal end of implantation needle 120 may be plugged with any conventional, biocompatible plug including, for example, bone wax, a silicone stopper, bioabsorbable material or the like, to prevent seeds and/or spacers from passing through and out of the distal end of implantation needle 120.

Implantation needle 120 may be connected to loader 100 via a stopper 124 provided with an axial cylindrical channel therein to permit insertion of the proximal end of implantation needle 120 into the distal end of stopper 124, to permit seeds and spacers to travel through the cylindrical channel in stopper 124 from holding area 110 to implantation needle 120, and to permit engagement of the proximal end of stopper 124 with the distal end of connector 126 for connecting stopper 124 to tip 118 of loader 100.

In one embodiment, actuators 104, 106 are designed to return to their original position, as a result of, for example, a biasing device such as springs 128, 130 operatively connected to actuators 104, 106 at one end and which rest against, for example, a shoulder 132 provided in housing 102 of loader 100. As shown in FIG. 5D, depression of actuator 104 compresses spring 128 against shoulder 132 to provide the required return force needed to return actuator 104 to its original position after a seed or spacer is dispensed. Depression of actuator 104 causes stylet 26 to enter the stylet opening in container 112 and force seed 20 from container 112 through the seed dispensing opening, as shown. Seed 20 falls into funnel 111 which is connected to holding area 110 to thereby direct seed 20 into the next available position in holding area 110. Similarly, seeds or spacers can be dispensed from container 114 by depression of actuator 106 in which case funnel 111 will collect the dispensed seed or spacer and direct it to holding area 110.

Also shown in FIG. 5D is that housing 102 defines a channel 136 that permits transfer of seeds and/or spacers from holding area 110 into a receiving device, as described above. Channel 136 is separated from holding area 110 by a closure such as a flap 134 for temporarily preventing transfer of seeds or spacers from holding area 110 to channel 136 until it is desired to transfer the seeds and spacers to another device. In operation, insertion of an implantation needle 120 into channel 136 will cause the proximal end of implantation needle 120 to push open flap 134 and thereby allow seeds and/or spacers located in holding area 110 to fall into implantation needle 120.

It is to be understood that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. A container for use in holding and dispensing one or more of brachytherapy seeds and brachytherapy spacers, said container comprising:
a housing that defines a holding area of sufficient size to house a plurality of brachytherapy seeds and brachytherapy spacers,
an advancing strip at least partially located in said holding area and in engagement with at least one of said brachytherapy seeds and brachytherapy spacers such that movement of said advancing strip in an indexing direction, causes movement of said brachytherapy seeds and brachytherapy spacers in the indexing direction, and
at least one dispensing opening in said housing of sufficient size to permit dispensing of one of a brachytherapy seed and a brachytherapy spacer from said holding area in said container through said opening in said housing.

2. A container as claimed in claim 1, wherein said advancing strip extends for a length sufficient for said advancing strip to contact every brachytherapy seed and brachytherapy spacer located in said holding area.

3. A container as claimed in claim 2, wherein said advancing strip includes a plurality of surfaces adapted for engagement with a surface attached to said housing, said surfaces of said advancing strip and said surface attached to said housing being oriented relative to each other such that engagement of one said surface of said advancing strip with the surface attached to said housing inhibits movement of said advancing strip in a direction opposite the indexing direction.

4. A container as claimed in claim 3, wherein said advancing strip comprises an engaging portion which engages at least one of said brachytherapy seeds and spacers in said holding area such that movement of said advancing strip in the indexing direction exerts a force on at least one of said brachytherapy seeds and spacers via said engaging portion to thereby index said brachytherapy seeds and spacers in the indexing direction and position a next seed or spacer for dispensing through said dispensing opening in said housing.

5. A container as claimed in claim 4, further comprising a stylet opening in said housing, said stylet opening positioned on an opposite side of said housing from said dispensing opening and in alignment with said dispensing opening.

6. A container as claimed in claim 5, wherein said surface attached to said housing and said surfaces of said advancing strip form part of a ratchet mechanism.

7. A container as claimed in claim 6, wherein said surface attached to said housing is a surface on a tab which is positioned such that a surface of said tab can engage with a surface of said advancing strip.

8. A container as claimed in claim 6, wherein said advancing strip further comprises a tab adapted for attachment to a pulling device for the purpose of exerting a pulling force on said advancing strip in the indexing direction to cause said advancing strip to advance a seed or spacer to a dispensing position.

9. A container as claimed in claim 8, wherein said housing comprises an advancing strip opening of sufficient size to permit said tab of said advancing strip and said advancing strip to extend outside of said housing.

10. A container for use in holding one or more of brachytherapy seeds and brachytherapy spacers, said container comprising:
a belt that defines a plurality of openings each of said openings being of sufficient size to house a single brachytherapy seeds or brachytherapy spacer.

11. A container as claimed in claim 10, wherein said belt comprises two layers, said layers being attached to one another between said openings and said layers being spaced from one another at defined intervals to thereby define said openings.

12. A container as claimed in claim 11, wherein a distance between any two adjacent openings in said belt is substantially constant.

13. A container as claimed in claim 12, wherein said belt is sufficiently flexible that said belt can be rolled up into a roll for storage.

14. A container as claimed in claim 13, wherein said container is bandoleer-shaped.

15. A container as claimed in claim 14, wherein said belt includes indicia to indicate a number of openings in said belt.

16. A hand-held brachytherapy seed-loading device, which comprises;
a housing sized for grasping in one hand by an adult human user,
a selection mechanism capable of selectively dispensing brachytherapy seeds or spacers from two or more sources of brachytherapy seeds or spacers, said selection mechanism being attached to said housing in a position whereby said selection mechanism can be actuated by one or more fingers of a hand grasping said housing,
a holding area for receiving said brachytherapy seeds or spacers dispensed by said selection mechanism,
a channel for transferring brachytherapy seeds or spacers from said holding area to another device, and
a closure for temporarily preventing transfer of brachytherapy seeds and spacers from said holding area to said channel to maintain seeds and spacers in said holding area until it is desirable to transfer said seeds and spacers to another device.

17. A hand-held brachytherapy seed-loading device as claimed in claim 16, wherein said holding area is provided with a window which permits viewing of contents located in said holding area.

18. A hand-held brachytherapy seed-loading device as claimed in claim 17, wherein said holding area is adapted to hold seeds or spacers in a linear, single-file arrangement in a same order as said seeds or spacers are selectively dispensed by said selection mechanism.

19. A hand-held brachytherapy seed-loading device as claimed in claim 18, wherein said housing is provided with contours to aid a user in gripping said housing with a single hand.

20. A hand-held brachytherapy seed-loading device as claimed in claim 19, wherein said selection mechanism comprises:
at least two actuators, and
at least two stylets operatively connected to said actuators.

21. A hand-held brachytherapy seed loading device as claimed in claim 20, wherein said selection mechanism further comprises an indexing device for indexing a device selected from an advancing strip and a belt to thereby position a next seed or spacer in position for dispensing by said selection mechanism.

22. A hand-held brachytherapy seed loading device as claimed in claim 21, further including at least two biasing devices, each said biasing device being operative engaged with one said actuator to bias said actuator to an original position upon dispensing of a seed or spacer to said holding area of said loading device.
